(19) United States
(12) Patent Application Publication
(10) Pub. No.: US 2003/0032896 A1
(43) Pub. Date: Feb. 13, 2003

(54) Microvolume embryo transfer system

(75) Inventors: Rodney W. Bosley JR., Bloomington, IN (US); Troy W. Wingler, Gosport, IN (US)

Correspondence Address: BRINKS HOFER GILSON & LIONE, P.O. Box 10395, Chicago, IL, 60610, US

(73) Assignee: Vance Products, Inc., d/b/a/ Cook Urological, Inc.

(21) Appl. No.: 10/264,651

(22) Filed: Oct. 4, 2002

Related U.S. Application Data
(63) Continuation-in-part of application No. 09/669,315, filed on Sep. 25, 2000.

(51) Int. Cl. A61B 5/00; A61D 7/00

(52) U.S. Cl. 600/585; 600/33

(57) ABSTRACT

An embryo transfer catheter that uses a very low transfer volume and a very soft tip is revealed. The use of a very small volume reduces the possibility of flushing and washing of the embryo from the uterus, while the very soft tip of the catheter reduces trauma to the endometrium in the event that the catheter is inserted too far into the uterus. There is an unexpectedly high rate of pregnancy in women receiving embryos from the new catheter.

### FIELD OF THE INVENTION

This application is a continuation-in-part of U.S. patent application Ser. No. 09/669,315, presently pending. This application claims the benefit of the filing date under 35 U.S.C. §120 of U.S. patent application Ser. No. 09/669,315, filed Sep. 25, 2000, which is hereby incorporated by reference in its entirety, and also claims the benefit of the filing date under 35 U.S.C. §119(e), of U.S. Provisional Application No. 60/156,049, filed Sep. 24, 1999, which is also hereby incorporated by reference in its entirety.

### BACKGROUND OF THE INVENTION

Human In Vitro Fertilization (IVF) and Embryo Transfer (ET), first successfully performed in 1978, has become a widely practiced procedure to treat infertile couples who have failed with more conventional methods of therapy such as superovulation and intrauterine insemination. The most common indications for IVF and related procedures, such as Gamete In Vitro Fertilization or Gamete Intra-Fallopian Transfer (GIFT), are women having blocked or damaged fallopian tubes. Other common indications include low sperm quality, or low egg quality, or both. Related factors include age of the female, and the degree of endometrial receptivity. The procedure may also be used in cases of severe male factor where direct (intracytoplasmic) injection of sperm is an option. Another indication for the procedure is when the shell of the egg is abnormally thick, thus preventing the fertilized and early-dividing embryo from escaping and implanting into the uterus. Creating a small opening through the shell has been shown to increase implantation rates. IVF is also being used when clinical or genetic factors require implantation of donor eggs from a fertile female that are fertilized in vitro and implanted into the recipient female using standard techniques.

The IVF/ET procedure typically involves the hormonal stimulation of the female to first suppress her ability to ovulate on her own, then stimulate development of follicles in the ovaries with a fertility medication. The mature eggs are removed from the ovary transvaginally using a needle, preferably guided under ultrasound. Following harvesting of the eggs, the eggs are identified and sorted with regard to maturity, and then placed with a sperm sample from the male. Approximately 24 hours after fertilization, the eggs are examined to confirm fertilization, which occurs in approximately 65% to 85% of the eggs harvested. After a short development period, the embryos are transferred, along with a volume of fluid, to the uterus using a delivery catheter. The delivery catheter is made of a soft plastic material to avoid damage to the endometrium.

There are many potential difficulties in achieving a successful implantation. Because of the soft nature of the standard delivery catheter, in a number of cases, the tip of the catheter may bend back on itself or curve away from the fundus of the uterus. The tip may also accidentally pass between the layer of the endometrium and myometrium. Conversely, a stiffer catheter increases the risk of trauma to the uterus or cervix, with the latter possibly leading to the release of prostaglandins and expulsion of the eggs from the endometrium.

Several unsuccessful attempts have been made to improve success rates. U.S. Pat. No. 6,165,165 uses a guiding catheter and an implant catheter, the implant catheter made from materials of two different durometers, so that the stiffness of the catheter decreases from the proximal end to the distal end of the catheter. While there may be some improvement in softness, there has been no dramatic improvement in success rates. Another problem is that there may be too much medium used in transferring the embryo from the transfer tube to the uterus. Even though the volume used to transfer may only be in the range of 30-50 microliters, this amount of medium may be sufficient to flush the embryo from the uterine cavity.

What is needed is a catheter system that can increase the likelihood of successful embryo implantation patients desiring this procedure without damaging the endometrium and without flushing the embryo from the uterus.

### SUMMARY

One embodiment of the invention is an embryo transfer system. The system comprises a guide catheter and a soft transfer catheter within the guide catheter, the transfer catheter having an inner diameter of about 0.015 inches (about 0.38 mm) to about 0.022 inches (0.56 mm). Another embodiment of the invention is an embryo transfer system comprising a guide catheter and a transfer catheter. The transfer catheter is configured for use within the guide catheter, the transfer catheter further comprising a thermoplastic polyolefin elastomer having a Shore A durometer hardness of about 85. Another embodiment of the invention is a method of implanting an embryo in a uterus. The method comprises passing a guide catheter through an opening in a cervix. The method then comprises passing a transfer catheter through the guide catheter, the transfer catheter having an inner diameter from about 0.015 inches (0.38 mm) to about 0.022 inches (0.56 mm). The method then comprises transferring an embryo in a medium from the transfer catheter, wherein a volume of the embryo and the medium transferred is from about five microliters to about ten microliters. More volume of fluid may also be used.

There are many ways to practice the present invention, as shown in the following drawings and specification. The embodiments described below are not meant to limit the invention, but rather to describe and illustrate the many ways that the present invention may be used.

### BRIEF DESCRIPTION OF THE FIGURES

FIG. 1 depicts a plan view of an embryo transfer catheter with a partial cross-sectional view.

FIG. 2 depicts a plan view of a guide catheter.

FIG. 3 depicts a plan view of an alternative embodiment of an embryo transfer catheter.

FIG. 4 depicts a reinforced embryo transfer catheter with a partial cross-sectional view.

FIG. 5 depicts an embodiment in which the guide catheter further comprises an echogenic tip.

FIG. 6 depicts an embodiment in which the transfer catheter is made from two materials.

FIG. 7 depicts an embodiment in which the transfer catheter has a step transition.

FIGS. 8 and 9 depict embodiments in which the guide and transfer catheters are combined into a single catheter.

FIGS. 10-13 are flowcharts for methods of practicing the invention.

### DETAILED DESCRIPTION OF THE PREFERRED EMBODIMENTS

FIGS. 1-2 depict an embryo transfer catheter system that comprises two catheters, an inner embryo transfer catheter 10, and a guide catheter 20.

The embryo transfer catheter 10 extends somewhat longer than the guide catheter 20. The inner catheter, the embryo transfer catheter 10 includes a passageway 13 of sufficient diameter to hold and deliver early embryos, gametes (oocyte or sperm), blastocysts, or zygotes that are to be transferred from in vitro culture for in vivo implantation and/or fertilization.

The embryo transfer catheter 10 includes a proximal portion 12 and a distal portion 14. The proximal portion may include a hub 15 for interfacing with the guide catheter. The catheter may also include an echogenic tip 16, preferably made of stainless steel, for detecting the distal end via ultrasound.

The catheter may also have markings 18 at the proximal or distal end indicating a position of the catheter to implantation personnel. Inner luminal surface 19 may be coated with a lubricious material or treated to reduce the surface energy density. A transfer catheter embodiment of the present invention may or may not have a coating or a treatment on its inner or outer luminal surface. The catheter itself is made of relatively soft material, such as polyethylene. Other materials that may be used include urethane, polyolefin, polyamides, polytetrafluoroethylene and silicone.

It has been found that a very soft material, having a Durometer reading of from about 80 to about 90 on a Shore A Durometer, is advantageous when used with an embryo transfer catheter having a narrow diameter and transferring a small volume of medium along with an embryo. To be useful as a delivery catheter, a soft material must be biocompatible and nontoxic to embryos. Thermoplastic olefin elastomers may be useful in these applications. Several grades of polyolefin may be useful for this purpose, including olefins made from blends of ethylene and octene. The soft grades are preferred, such as Engage® 8100 and 8480, especially preferred is 8003, which has a slightly higher density than 8100 and 8480.

The diameter of the passageway and volume of the fluid and material contained therein is preferably minimized to a diameter of no greater than 0.022″, and most preferably between 0.015″ and 0.022″. The transfer volume is no greater than 15 microliters, preferably between 5 and 10 microliters. The outer diameter of the delivery catheter is preferably 4.4 Fr (about 0.058 inches), although other diameters may be used. The delivery catheter preferably has a length of from about 18 to 25 cm, with lengths of 18.5 cm and 23.5 cm particularly preferred. These lengths are typically referred to in the industry as “19 cm” and “24 cm,” respectively. Other lengths may be used. Clinical experience with this catheter for IVF/ET having a 0.020″ inner diameter with a volume of approximately 10 microliters indicates an unexpected increase in pregnancy rates, up to eighty percent success rates, possibly due to reduced trauma and the reduced amount of fluid delivered with the embryos. This success rate was achieved with one clinical study group having about forty patients. The reduced transfer volume ostensibly lessens the tendency of embryos to migrate to another section of the uterus, for instance, into the fallopian tubes. By increasing the implantation rate, fewer embryos may be needed, thereby reducing the number of unwanted multiple pregnancies and further risks.

FIG. 2 depicts a guide catheter 20 used coaxially with the embryo transfer catheter 10. Guide catheter 20 is of relatively simple construction, and may comprise a proximal portion 22 and a distal portion 24, and distal end 26. Distal end 26 is preferably open rather than closed. The catheter 20 also comprises a hub 25 for interfacing with the embryo transfer catheter 10. The guide catheter may also comprise markings 28 at distal portion 24 to guide delivery personnel. The guide catheter is preferably somewhat stiffer than the embryo transfer catheter. Materials suitable for the guide catheter are many, so long as the guide catheter is able to hold its shape without drooping or sag during the implantation procedure. Materials which have been used include polytetrafluoroethylene (PTFE), although other materials, as mentioned above, may also be used. The guide catheter has a diameter that allows the delivery catheter to comfortably pass without restriction. The guide catheter is preferably about 12 to about 17 cm in length, and is preferably 6.8 Fr (about 0.088 inches). Other lengths and diameters may be used.

The embryo transfer catheter and the guide catheter are used to implant an embryo into a uterus of a woman. The echogenic tip and the markings on one or more of the catheters assist in this operation, to guide operating personnel as to its exact position and to complete the embryo transfer procedure. It is preferable to use a syringe and to transfer the embryo into the uterus by means of fluid pressure.

As mentioned above, optional graduated markings 18, 28 can be placed about proximal portions 12 of delivery catheter 10 or distal portion 24 of guiding catheter 20 to determine the depth of penetration into the uterus or the amount of delivery catheter 10 that is exposed beyond distal tip 26 of guiding catheter 20. Additional graduated markings may also be placed on the guide catheter if desired.

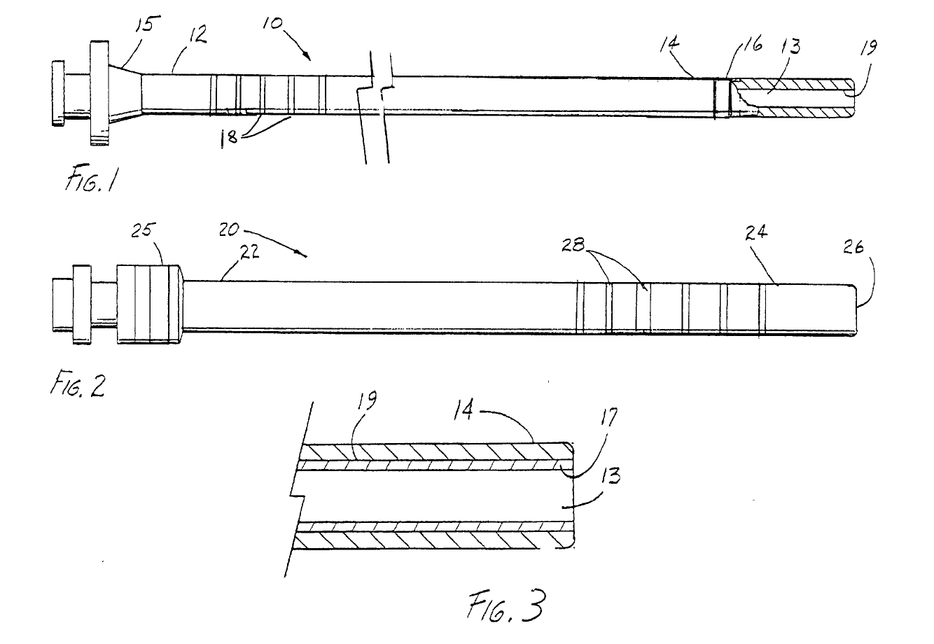

Because the delivery catheter is preferably made of a softer (low durometer) polymer, the surface energy density (wetting ability) may be higher, making the delivery fluid and embryo more likely to adhere to the inner luminal surface. This is especially critical because there is an increased likelihood of problems in delivery with a catheter having a reduced lumen diameter. A typical oocyte has a diameter of about 100 microns (about 0.004 inches) and a blastocyst has a diameter of about 130 microns (about 0.005 inches). Luminal surface treatments may help reduce the wetting ability of the inner luminal surface and thus adherence to the inner luminal surface, resulting in the smooth expulsion of oocytes and embryos. Ion beam bombardment is a well-known technique for reducing surface energy density of polymers. Polishing and surface coatings can also offer improvement in friction coefficients for otherwise “sticky” polymers. FIG. 3 depicts an embodiment in which the inner luminal surface 19 of the passageway 13 of the distal portion 14 of the delivery catheter is coated with conformal material 17, such as parylene, to reduce surface energy density. Paralyne coatings may be applied by in-house systems or by vendors, such as Specialty Coating Systems, Indianapolis, Ind., or Parylene Coating Services, Katy Tex., among other vendors. Other coatings, such as those that impart a hydrophilic surface or a hydrophobic surface, may also be used. Such coatings may include, but are not limited to, silicone treatments. The outer surface of the catheter may also be coated or treated.

In addition to the delivery catheter embodiment depicted in FIG. 1, the embryo transfer catheter can be made with a stiffened proximal component. FIG. 4 depicts an embryo transfer catheter 40 having a stiffening or reinforcing portion 47 in proximal portion 42. Embryo transfer catheter 40 also includes a central lumen 43 and a distal portion 44, preferably with an echogenic tip 46. The echogenic tip may be made of stainless steel, including indentations, grooves, or other features to enhance echogenicity. The echogenic tip may also take the form of particles embedded into the outer surface of the catheter. It has been found that spherically-shaped metallic particles are better for the resulting ultrasonic images. The particles are preferably incorporated into the desired location of the embryo transfer catheter, or possibly into the guide catheter, by molding them into the catheter.

The proximal portion 42 may also includes graduated markings 48 and an interface 45. The reinforcing member may be a stainless steel tube that is bonded to the embryo catheter, preferably by heat or by an adhesive. However, the fit between the reinforcing member and the delivery catheter is typically sufficient that bonding is not required. The reinforcing member may be a cannula on the inside or on the outside of the transfer catheter. An example of a stiffened embryo transfer catheter is polyethylene tubing having a central lumen of 4.4 Fr diameter with a 23GXTW stainless steel cannula. An outer cannula, in which the polyethylene tubing is on the inside of the cannula, may also be used.

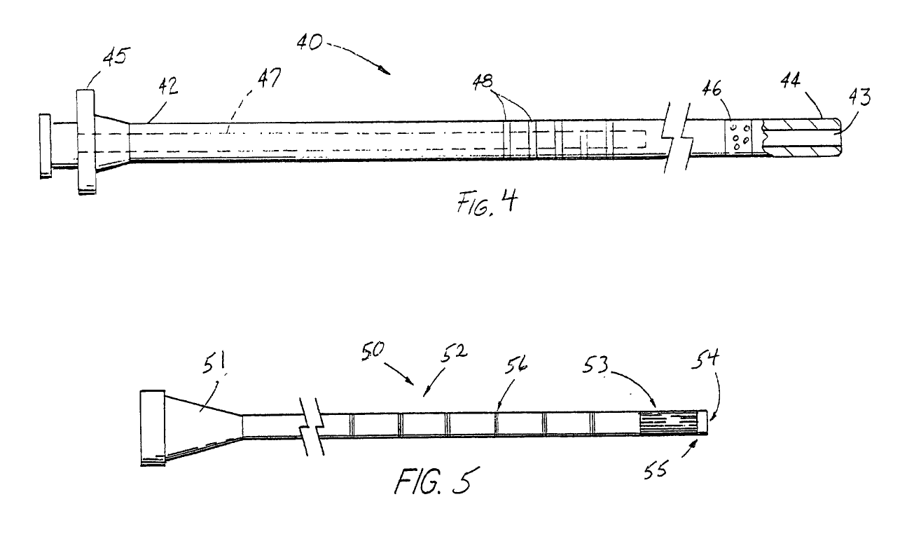

While it is advantageous for the transfer catheter to have an echogenic tip, it may also be desirable for the guide catheter to have such an echogenic tip. FIG. 5 depicts a guide catheter 50 having a proximal portion with connector 51 and a distal portion 52 and an echogenic tip 53 placed proximal to a distal tip 55. An echogenic tip may be placed as desired on either the guide catheter or the transfer catheter. The “tip” may be placed at either the proximal end or the distal end, or in between, depending on the preferences and needs of the surgical team accomplishing the embryo transfer. It is understood that the tip is not movable and that the tip may not be moved once it is manufactured or bonded to the guide catheter or the transfer catheter. The guide catheter may also have an internal passageway 54 to accommodate the transfer catheter. The guide catheter may also have markings 56 to help guide the surgeon in its placement.

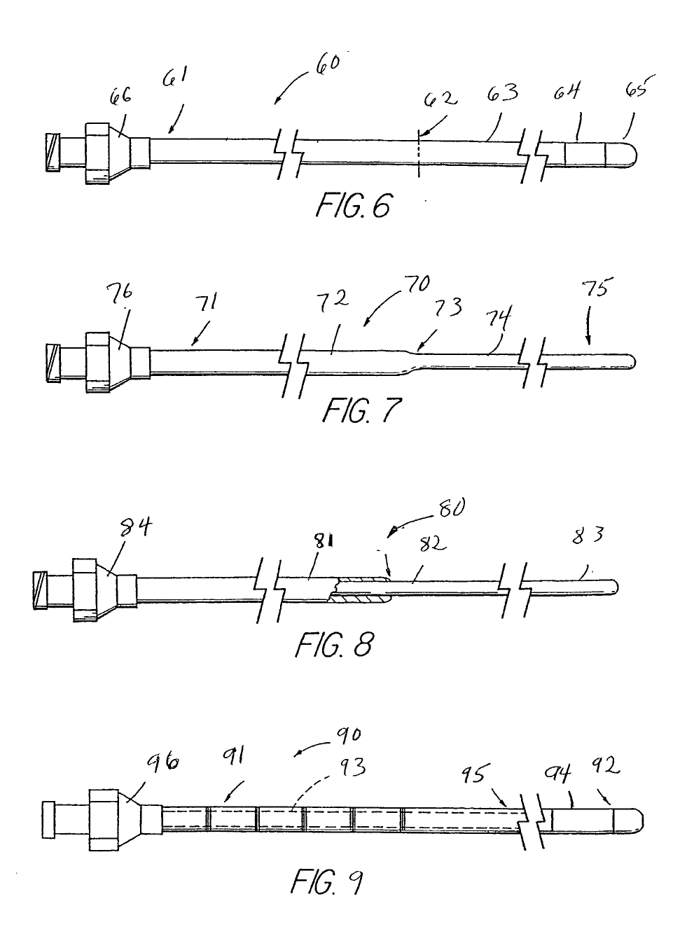

The transfer catheter may conveniently be made from a single material, as discussed above. The transfer catheter is not limited to a single material, however, and may be made as a composite of more than one material, as shown in FIGS. 6 and 7. FIG. 6 depicts a transfer catheter 60 made from a proximal portion 61 of a first material, and a distal portion 63 made from a second, softer material. There is a transition zone or transition area 62 where the first material ends and the second material begins. Transfer catheter 60 may also have an echogenic tip 64 just proximal to the distal tip 65 of the catheter. Catheter 60 may also have a fitting 66 molded or bonded to proximal portion 61 of the catheter. Another embodiment of a two-material catheter is depicted in FIG. 7. In this embodiment, transfer catheter 70 has a proximal portion 71 made from a first, harder or stiffer material, while the distal portion 74 is made from a softer material. At a desired point 73 in the distal portion, the catheter transitions from a larger, thick-walled catheter portion 72 to a smaller, perhaps thinner-walled, catheter portion 74. The material in distal portion 74, and particular in the distal tip 75, is softer and less stiff than the material used in the proximal portion of the catheter.

Instead of using two separate catheters, such as separate guide and transfer catheters, their functions may be combined into a single catheter, as shown in FIGS. 8 and 9. FIG. 8 depicts a single catheter 80 having the functions of a guide catheter and a transfer catheter. Guide catheter portion 81 is configured with a larger diameter to be stiffer and somewhat harder than transfer catheter portion 82, which has a smaller diameter. Distal tip 83 is desirably very soft, preferably from about Shore A 80 to about Shore A 90 durometer hardness. A connector 84 may also be bonded to the catheter. In many cases, it is difficult to accurately gauge the softness of a material with a certainty less than about five durometer points, so a distal tip desirably has a hardness reading of about 85 plus or minus five points, on a Shore A scale. Catheter 80 may be fabricated by separately molding two components and joining them together, or may be made as a single component, for instance, by co-extruding an inner, softer material and an outer, harder, stiffer material. FIG. 9 is another embodiment in which a single catheter 90 combines the functions of a guide catheter and a transfer catheter in a catheter having a single outer diameter for most of its length. The proximal portion 91 may include an inner, stiffer material component 93 and an outer, softer material 95. The distal portion 92 may include only the outer softer material. Catheter 90 may also include an echogenic tip 94 at its distal end as discussed above, and may have a connector 96 bonded or molded to its proximal end.

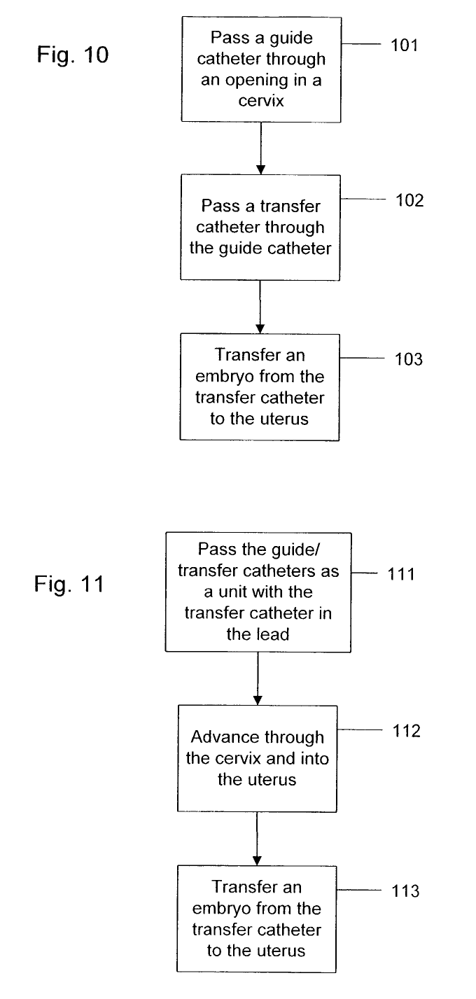

As discussed above, the transfer catheter and the guide catheter may be used to implant an embryo. Another embodiment of the invention is a method of using an embryo transfer system to implant an embryo in a uterus. FIG. 10 is a flowchart depicting one method. In this method, a first step is to pass 101 a guide catheter through an opening in a cervix. A transfer catheter is then passed 102 through the guide catheter, exposing the transfer catheter to the uterus. The method then includes transferring 103 an embryo from the transfer catheter to the uterus. The transfer is preferably accomplished by fluid pressure, using a small amount of fluid to accomplish the transfer.

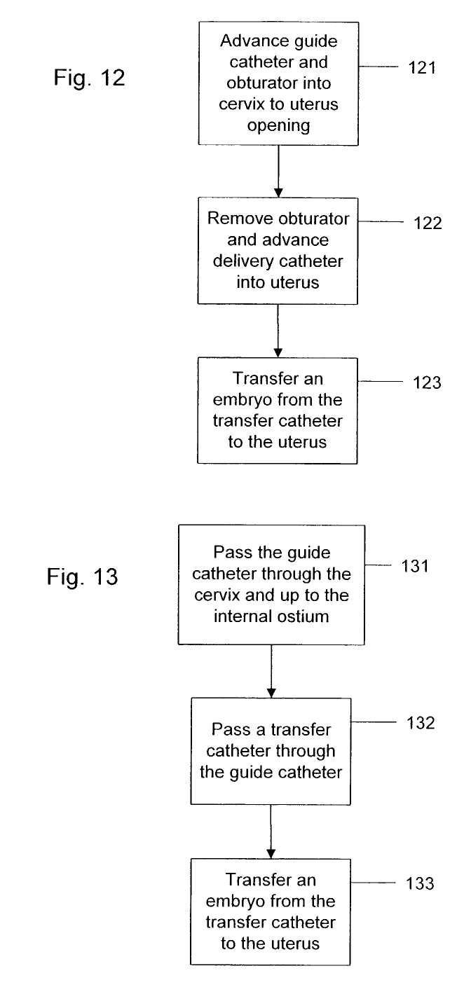

Other methods may be used with the microvolume transfer catheter embodiments of the present invention. In one method, as shown in FIG. 11, the guide catheter and the transfer catheter are passed 111 as a unit with the transfer catheter in the lead. The unit is advanced through the cervix and into the uterus 112. The method then includes transferring 113 an embryo from the transfer catheter to the uterus. The transfer is preferably accomplished by fluid pressure, using a small amount of fluid to accomplish the transfer. Another method is depicted in FIG. 12. A first step 121 of the method is to advance a guide catheter and an obturator into the cervix and to the opening of the uterus. An obturator, which may be malleable, is a device that fills and may occlude the central lumen, allowing the lumen and the guide catheter to better hold a specific shape during the procedure. The obturator is then removed and a delivery or transfer catheter is then advanced into the uterus 122 through the guide catheter. The method then includes transferring 123 an embryo from the transfer catheter to the uterus. The transfer is preferably accomplished by fluid pressure, using a small amount of fluid to accomplish the transfer. Another method is depicted in FIG. 13. The method includes passing 131 the guide catheter through the cervix and up to the internal ostium. The method includes passing a transfer catheter through the guide catheter 132. The method then includes transferring 133 an embryo from the transfer catheter to the uterus. The transfer is preferably accomplished by fluid pressure, using a small amount of fluid to accomplish the transfer.

The details of the construction or composition of the various elements of the cellular transfer catheter not otherwise disclosed are not believed to be critical to the achievement of the advantages of the present invention, so long as the elements possess the strength or flexibility or softness needed for them to perform as disclosed. The selection of such details of construction is believed to be well within the ability of one of even rudimentary skills in this area, in view of the present disclosure, and are within the spirit of the invention and the scope of the claims. It will be understood that no limitation of the scope of the invention is intended by the above description and drawings, which is defined by the claims below.

What is claimed is:

1. An embryo transfer system, comprising:
a guide catheter; and
a soft transfer catheter within the guide catheter, wherein the transfer catheter has an inner diameter of from about 0.015 inches (0.38 mm) to about 0.022 inches (0.56 mm).

2. The system of claim 1, wherein a distal end of the transfer catheter is more flexible than a proximal end of the transfer catheter.

3. The system of claim 1, further comprising a stiffening component in at least one of the guide catheter and the transfer catheter.

4. The system of claim 1, further comprising an echogenic tip in at least one of the guide catheter and the transfer catheter.

5. The system of claim 4, wherein material for the echogenic tip is selected from the group consisting of a metallic component and particles embedded within the transfer catheter.

6. The system of claim 1, wherein the transfer catheter further comprises at least one material selected from the group consisting of polyethylene, urethane, polyolefin, polyamide, silicone, poly-octene-ethylene, a thermoplastic polyolefin elastomer, and polytetrafluoroethylene, said at least one material having a Shore A Durometer hardness from about 80 to about 90.

7. The system of claim 1, wherein the transfer catheter comprises a poly-octene-ethylene material having a Shore A Durometer hardness of about 85.

8. The system of claim 1, wherein the transfer catheter has an inner diameter from about 0.018 inches (0.45 mm) to about 0.022 inches (0.56 mm).

9. The system of claim 1, wherein the transfer catheter is configured for a transfer volume of from about 5 microliters to about 10 microliters.

10. The system of claim 1, wherein the transfer catheter and the guide catheter are made as a single unit.

11. An embryo transfer system, comprising:
a guide catheter; and
a transfer catheter configured for use within the guide catheter, the transfer catheter further comprising a thermoplastic polyolefin elastomer having a Shore A durometer hardness of about 85.

12. The system of claim 11, wherein the transfer catheter has an inner diameter of from about 0.015 inches (0.38 mm) to about 0.022 inches (0.56 mm).

13. The system of claim 11, wherein the transfer catheter is configured for a transfer volume of from about five microliters to about ten microliters.

14. The system of claim 11, further comprising an echogenic tip on at least one of the guide catheter and the transfer catheter.

15. The system of claim 11, wherein the transfer catheter and the guide catheter are made as a single unit.

16. A method of implanting an embryo in a uterus, the method comprising:
passing a guide catheter through an opening in a cervix;
passing a transfer catheter partly through the guide catheter, the transfer catheter having an inner diameter from about 0.015 inches (0.38 mm) to about 0.022 inches (0.56 mm); and
transferring an embryo in a medium from the transfer catheter, wherein a volume of the embryo and the medium transferred is from about five microliters to about ten microliters.

17. The method of claim 16, wherein the transferring is accomplishing by fluid pressure from the transfer catheter.

18. The method of claim 17, wherein the transferring is accomplished by means of an echogenic tip on at least one of the guide catheter and the transfer catheter.

19. The method of claim 16, wherein the transfer catheter is first passed through the guide catheter, and the guide catheter and transfer catheter are advanced as a unit through an opening in the cervix with the transfer catheter in front of the guide catheter.

20. The method of claim 16, wherein the guide catheter is passed through the cervix and up to an internal ostium before the step of passing the transfer catheter at least partly through the guide catheter.

21. The method of claim 16, wherein the step of passing the guide catheter through an opening in a cervix comprises advancing the guide catheter and an obturator into the cervix to an opening in the uterus, and further comprises a step of removing the obturator before the step of passing the transfer catheter partly through the guide catheter.
